(19) United States
(12) Patent Application Publication
(10) Pub. No.: US 2024/0050769 A1
(43) Pub. Date: Feb. 15, 2024

(54) BREAST IMMOBILIZATION DEVICE

(71) Applicant: Patricia H Hardenbergh, Edwards, CO (US)

(72) Inventor: Patricia H Hardenbergh, Edwards, CO (US)

(21) Appl. No.: 18/034,709

(22) PCT Filed: Oct. 30, 2021
(86) PCT No.: PCT/US2021/057476

§ 371 Date: Apr. 30, 2023

Related U.S. Application Data
(60) Provisional application No. 63/107,509, filed on Oct. 30, 2020.

(51) Int. Cl. A61B 90/17

(52) U.S. Cl. CPC A61B 90/17; A61N 2005/1097

(57) ABSTRACT

The present disclosure is directed to a device for immobilizing a breast or breasts during radiation or other procedures and a method of use thereof. The device provides for accurately and repeatedly positioning a subject's breast away from the torso of the subject during treatment. The device includes at least one indexed rail secured alongside a radiation couch, at least one curvilinear, preferably nonarticulating, arm detachably secured to the at least one indexed rail, a bar comprising a hooked tension cord and a tensioner assembly element, and at least one breast engagement plat functionally and removably coupled to the at least one arm via the at least one bar.

### RELATED APPLICATIONS

This application claims the benefit of priority from U.S. Provisional Patent Application No. 63/107,509, filed on Oct. 30, 2020, the disclosure of which is hereby incorporated by reference in its entirety.

### FIELD OF INVENTION

The present invention relates generally to medical immobilization devices, and in particular, a device useful for the immobilization of a breast or breasts, or other body part(s), for the delivery of radiation therapy and other therapies or procedures.

### BACKGROUND

Breast cancer afflicts both men and women worldwide. Radiation therapy is frequently used as part of the standard treatment for various breast cancers. Such therapy may involve delivery to the whole breast or to a specifically identified area of a breast.

Acute radio-dermatitis is a significant complication ranging from mild to severe erythema, dry or moist desquamation, skin ulceration, and tissue necrosis. Additional complications may also arise from inadvertent or unavoidable radiation exposure of body organs, e.g., lungs, heart, and liver, in an attempt to treat the breast. Therefore, positioning the breast during radiation treatment is an on-going challenge in all shapes and sizes of breasts, and in particular, in breasts having larger volume and/or less support, i.e., large and/or pendulous breasts. Ptosis or sagging of breasts is a natural consequence of aging, gravity, body mass, and various other attributes, or a combination thereof. The plethora of breast size, shape, density, and degree of ptosis may interfere with and exacerbate attempts to accurately and repeatably dose radiation while avoiding unnecessary skin and organ involvement. For example, pendulous breasts may shift toward the abdomen of the subject or more importantly with respect to the goal of organ sparing, laterally toward the side of a subject.

Heretofore, various attempts have been made to more accurately provide radiation treatment and/or reduce the incidence of exposing organs other than the intended breast to radiation, for example, radiation with modulated intensity, patient positioning, and various devices for subject and breast immobilization. Standard radiation beams are delivered through tangential beams which are angled medial and lateral to the breast and therefore the depth of the radiation fields are dependent upon positioning of the breast to minimize the necessity to treat deep into the lung and heart due to the draping of a large breast to the side of the subject.

It would be desirable to provide a breast immobilization device and methods that provide comfortable and stabilized subject and breast positioning, and in turn, reproducible treatment, without sacrificing treatment capabilities. The reduction of unnecessary radiation exposure without sacrificing treatment capability or the enhancement of treatment capability and accuracy is an ongoing goal in medicine and medical procedures, in particular oncology. Thus, devices and methods that are effective to reduce inadvertent radiation exposure of organs (such as the heart and lungs) and immobilize the breast or breasts in a discrete position while creating reproducible treatment would improve the quality of life, improve treatment accuracy and repeatability, and reduce adverse impact of incidental or inadvertent organ radiation exposure of a subject.

### SUMMARY

This disclosure relates to a device for immobilizing a subject's body part, e.g., breast, during radiation therapy, comprising at least one support arm component coupled to a radiation couch, at least one breast engagement plat component, and at least one bar component functionally coupling the at least one support arm component to the at least one breast engagement plat. The present disclosure has various aspects. These aspects provide, for example, a device and methods relating to immobilization of breasts, in particular, in a position away from the subject's thorax, i.e., lifts the breast, during treatment and/or procedures, e.g., radiotherapy, not previously known.

In one aspect, the disclosure provides a device for supporting and stabilizing, i.e., immobilizing, a breast during radiotherapy. Ideally, the device and subject should remain in a fixed relationship, wherein the subject's body part is consistently immobilized in a predetermined position for accurate and repeatable treatment. In some embodiments, the device comprises at least one curvilinear support component (“arm”) having a first fixed end and a free second end, wherein the first fixed end is coupled to a radiation table (“couch”), a breast engagement plat component (“plat”), and at least one, and preferably a plurality, bar coupling component (“bar”) functionally coupling the plat component to the arm component, wherein the bar is comprised of at least one tension cord and at least one tensioner assembly element. In some embodiments, the fixed end of the at least one support arm may be coupled to a radiation table indexing rail (“rail”) mounted lengthwise and horizontally planar to the couch. In some embodiments, the fixed end of the at least one support arm may be coupled directly to a radiation couch. The at least one support arm component may be secured to a table in a discrete position, and thus, set the bar component(s) at a desired position and orientation with relation to the subject.

In some embodiments, the at least one support arm component may comprise a curvilinear support arm having a first fixed end and a second fixed end, wherein each of the first and second ends is secured to an opposing side of a couch or opposing generally parallel indexing rails mounted lengthwise and horizontally planar to the couch.

In some embodiments, the at least one support arm component may comprise a structure having, e.g., at least one discrete angle, for example, acute, right, or obtuse. In some embodiments, the at least one support arm component may comprise one or more semi-circle, hoop-like, or “C” structures.

In some embodiments, the at least one support arm component may carry a plurality of indicia corresponding to support arm component apertures therethrough, either horizontal or vertical with respect to the plane of a radiation table. In some embodiments, the at least one support arm component may carry a plurality of indicia indicative of discrete positions on the at least one support arm component and useful, for example, for tensioner assembly element placement. In some embodiments, the at least one support arm component may carry a plurality of indicia corresponding to support arm component apertures and identifying discrete positions thereon. Indicia and configurations of the present disclosure are intended to facilitate reproducible subject positions, subject breast positions, and in turn, radiation treatment accuracy and repeatability.

Some embodiments may further comprise the use of traceable and/or trackable markers, i.e., indicia, tattoos, etc., incorporated as part of the present device and method and may be useful for, e.g., supplemental positioning accuracy and reproducibility.

The disclosed device provides subject breast support resulting in an improved breast position that minimizes or eliminates organ radiation exposure and/or enhances the reproducibility of radiation treatment of a breast. The reduction or elimination of organ, e.g., heart, lung, irradiation, in turn, may reduce the side effects of radiation therapy. The support arm component shape and/or curvature, size, and adjustable positioning allows for accommodation of a broad range of size and shapes of breasts and subjects.

Another aspect of the disclosure provides a method for use of the device. The method comprises fitting a subject with at least one breast engagement plat component, positioning the subject on a radiation table or a support board in a supine orientation, positioning and attaching at least one support arm component to the radiation table or support board, removably coupling at least one bar to the at least one support arm component, functionally coupling the at least one bar to the at least one breast engagement plat, and optionally adjusting the at least one bar.

Another aspect of the disclosure provides a method of medical treatment which involves support of a subject's breast using any embodiment of the device described herein and, while supporting the subject's breast, delivering radiation to the subject.

In a preferred embodiment, a device for immobilizing at least one breast of a radiation therapy subject comprising at least one support arm component, at least one breast engagement plat; and at least one bar component detachably engaging the at least one support arm component to the at least one breast engagement plat is provided.

In another preferred embodiment, a device for immobilizing at least one breast of a radiation therapy subject comprises at least one indexing couch rail secured lengthwise and in parallel plane to the radiation couch; at least one support arm component comprising a curvilinear arm having a first fixed end and a second free end, wherein the first fixed end is detachably fixed to the at least one indexing couch rail: at least one bar component comprising at least one tension cord and at least one tensioner assembly element, wherein the at least one tension cord has a first end having a hook element disposed thereon and a second and opposite free end, and the at least one tensioner assembly element is movably mounted on the at least one support arm; at least one breast engagement plat comprising a top surface and bottom surface, wherein at least one bar engagement element is disposed on the top surface; and the first end of the at least one bar tension cord detachably couples to the at least one bar engagement element of the breast engagement plat and the second free end of the tension cord detachably couples to the at least one tensioner assembly element.

In some embodiments, the at least one indexing couch rail further comprises a plurality of apertures therethrough, wherein the apertures may be smooth bores, threaded bores, splined bores, or a combination thereof.

In some embodiments, the support arm component comprises a non-articulating support arm.

In some embodiments, the support arm component comprises a plurality of substantially parallel indexed arm support rails defining a space therebetween adapted for receiving at least one tensioner assembly element.

In some embodiments, the support arm component comprises two fixed ends.

In some embodiments the support arm component may further comprise a plurality of apertures therethrough.

In some embodiments, the support arm component comprises a plurality of segments adapted for interlocking engagement.

In some embodiments, the device may further comprise indexing indicia.

In some embodiments, the at least one breast engagement plat comprises at least a top surface and a bottom surface comprised of a single layer or a plurality of layers of construction material, wherein the top surface may further comprise at least one bar engagement element disposed thereon and the bottom surface is configured to couple to the breast of a subject.

In some embodiments, the at least one tension cord is adapted for direct engagement with the at least one support arm.

In some embodiments, the tensioner assembly element is selected from spring activated, spooling, cleated, and spooling and cleated.

In some embodiments, a device and method for immobilizing at least one breast of a radiation therapy subject comprises at least one support arm component comprising a curvilinear arm having a first fixed end and a second free end, wherein the first fixed end is detachably fixed to a treatment couch.

In some embodiments, a method of using a device for immobilization of a breast, the method comprising the steps of: securing at least one indexing rail to a radiation couch; attaching a breast engagement plat having at least one loop disposed thereon to a subject; arranging a subject in a supine position on the radiation couch: securing at least one support arm to the at least one indexing rail or to the radiation couch; mounting at least one tensioner assembly element to the at least one support arm; functionally engaging the at least one loop of the breast engagement plat with a tension cord comprising a first hooked end and a second free end; and, adjustably coupling the second free end of the tension cord to the at least one tensioner assembly element, is provided. Some embodiments of the method further comprise adjusting the components of the device in relation to the subject.

The above and other feature, advantages, and aspects will be apparent from the exemplary embodiments illustrated in the accompanying drawings and/or described in the embodiments of the detailed description. The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate non-limiting examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIGS. 1A-B are perspective views of an exemplary embodiment of a breast immobilization device according to the present disclosure: FIG. 1A shows an embodiment wherein a support component comprises at least one non-articulating, curvilinear arm having a fixed end and a free end; and, FIG. 1B shows a schematic illustration of a radiation delivery method from multiple radiation beam angles to a subject's breast when immobilized by the device of the present disclosure.

FIGS. 2A-D are perspective views of additional exemplary embodiments of the present disclosure: FIG. 2A shows a breast immobilization device according to the present disclosure, wherein the support component comprises a curvilinear arm terminating in one fixed end and an opposite free end configured to receive a tubular tensioner assembly element; FIG. 2B shows a breast immobilization device according to the present disclosure, wherein the support component comprises a curvilinear arm comprised of two generally parallel elements defining an equidistant curvilinear space there between; FIG. 2C shows a breast immobilization device according other present disclosure, wherein the support component comprises a curvilinear arm terminating in two opposing fixed ends and a plurality of apertures therethrough configured to receive a tensioner assembly element or tension cord; and, FIG. 2D shows a breast immobilization device according to the present disclosure wherein the support component comprises a curvilinear arm terminating in two opposing fixed ends comprised of two generally parallel elements defining an equidistant curvilinear space there between.

FIGS. 3A-D are isolated views of a support arm component configuration of the present disclosure and a preferred exemplary embodiment of bar components, i.e., tension cord and tensioner assembly element, of the present disclosure: FIG. 3A shows a first view of a support component arm having mounted thereon a plurality of tubular tensioner assembly elements, a plurality of breast plats, and a plurality of bars comprised of a plurality of tension cords functionally coupling the breast plat(s) to the tensioner assembly element(s); FIG. 3B shows a reverse view of the support arm component of FIG. 3A comprised of segments adapted for interlocking engagement: FIG. 3C shows an isolated view of an alternate embodiment of a support arm component with a spool tensioner assembly element mounted thereon; and, FIG. 3D is an isolated view of an embodiment of a tension cord having a first hooked end and an opposite free end.

FIG. 4 shows an exploded and isolated view of a spline indexing embodiment of the disclosure, wherein a first fixed end of a support arm comprises a spline insertion element and a corresponding indexing rail component is comprised of at least one spline receiving aperture.

FIGS. 5A-G show exemplary configurations of the breast engagement plat component of the present device, wherein each embodiment evidences alternate plat configurations adapted to provide individual and different breast support and immobilization characteristics.

FIGS. 6A-B show isolated views of exemplary embodiments of breast engagement plat component construction: FIG. 6A shows a multilayer plat exemplary embodiment; and, FIG. 6B shows a single layer plat exemplary embodiment.

FIGS. 7A-C are isolated views of exemplary embodiments of tensioner assembly elements of the present disclosure: FIG. 7A shows a tubular spring actuated tensioner assembly element; FIG. 7B shows a push button spool actuated tensioner assembly element; and, FIG. 7C shows a spring and spool actuated tensioner assembly element.

FIGS. 8A-B are views of a rail engagement system of the disclosure comprising an adjustable clamp element; FIG. 8A is a perspective view of an adjustable clamp engaged with an indexing couch rail of the present disclosure; and, FIG. 8B is an isolated view of an exemplary clamp element.

FIGS. 9A-B are isolated views of an exemplary modular rail component embodiment of the present disclosure: FIG. 9A shows an exploded view of a modular alternate embodiment; and, FIG. 9B shows an integrated view of the modular alternate embodiment of the rail component.

### DETAILED DESCRIPTION

Broadly, the present disclosure contemplates a breast immobilization device and method, having a construction and configuration that decreases radiation exposure of the organs of a subject and enhances the accuracy and reproducibility of radiation treatment, and thus, minimizes unwanted radiation and side effects. When practiced as disclosed herein, the present disclosure provides a novel and effective breast immobilization device and methodology useful as disclosed and exemplified herein but not intended to be limited to these uses.

The embodiments of the device described herein provide exemplary embodiments only, and are not intended to be exhaustive, limit the scope, applicability or configuration of the device. Rather, the specific details of the disclosure are set forth to provide a thorough understanding to those skilled in the art with an enabling description for implementing one or more of the exemplary embodiments. It is understood by those skilled in the art the device may be practiced without these specific details and that that various changes may be made, e.g., in the function and arrangement of elements, without departing from the spirit and scope of the disclosure as set forth. In some instances, well known elements may not be shown or described to avoid obfuscation of the device.

Unless specifically noted, it is intended that the words and phrases in the specification and the claims be given their plain ordinary and accustomed meaning to those of ordinary skill in the applicable arts, and as used herein are provided as non-limiting examples of the features of the invention. Accordingly, various implementations may be very broadly adopted and applicable.

The present disclosure relates generally to immobilization devices used during radiation treatment and other procedures. Described herein, in particular, the present disclosure provides examples of immobilization device configuration, examples of device materials, and systems and methods for using same. The device of the present disclosure provides immobilization via novel configuration for improved treatment and reduced side effects.

Each of the components comprising the presently disclosed device and related method may be constructed in various manners, e.g., machining, casting, molding, 3D printing, heat formed, joined, fastened, die cut, or other fabrication methods now known or known in the future that would produce components with appropriate attributes, e.g., size, shape, strength, weight.

As used herein, “at least one”, “one or more”, and “and/or” are open-ended expressions that are both conjunctive and disjunctive in operation. For example, each of the expressions “at least one of A. B and C”, “at least one of A, B, or C”, “one or more of A, B, and C”, “one or more of A, B, or C” and “A, B. and/or C” means A alone, B alone, C alone, A and B together, A and C together, B and C together, or A, B and C together. When each one of A, B, and C in the above expressions refers to an element, such as X, Y. and Z, or class of elements, such as X₁-Xₙ, Y₁-Yₘ, and Z₁-Zₒ, the phrase is intended to refer to a single element selected from X. Y, and Z, a combination of elements selected from the same class (for example, X₁ and X₂) as well as a combination of elements selected from two or more classes (for example, Y₁ and Zₒ).

It is to be noted that the term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “means” as used herein shall be given its broadest possible interpretation in accordance with 35 U.S. C. Section 112(f). Accordingly, a claim incorporating the term “means” shall cover all structures, materials, or acts set forth herein, and all of the equivalents thereof. Further, the structures, materials, or acts and the equivalents thereof shall include all those described in the summary, brief description of the drawings, detailed description, abstract, and claims themselves.

It should be understood that every maximum numerical limitation given throughout the present disclosure is deemed to include each and every lower numerical limitation as an alternative, as if such lower numerical limitations were expressly written herein. Every minimum numerical limitation given throughout the present disclosure is deemed to include each and every higher numerical limitation as an alternative, as if such higher numerical limitations were expressly written herein. Every numerical range given throughout the present disclosure is deemed to include each and every narrower numerical range that falls within such broader numerical range, as if such narrower numerical ranges were all expressly written herein.

A preferred embodiment of the device of the present disclosure comprises at least one indexing couch rail secured lengthwise and in a parallel plane to a radiation couch, at least one curvilinear, preferably non-articulating, arm removably secured to the at least one couch rail, at least one bar element comprising a hooked tension cord and a tensioner assembly element, and at least one breast engagement plat functionally and removably coupled to the at least one support arm via the at least one bar element.

### Indexing Rails

Indexing rail or rails of the present disclosure are useful in conjunction with a treatment. e.g., radiation, couch or table having opposite sides to which at least one indexing rail may be removably, semi-permanently, or permanently secured. The rail or rails may be secured, for example, via clamping means, via construction as part of the couch, i.e., flanges or extensions onto which a rail may be mounted, or via an independent indexing rail module couch overlay. Radiation couches are well known by those skilled in the art and therefore, the rails of the disclosure are intended to be easily adapted to integrate therewith. Indexing rail embodiments employing a clamping means of attachment may comprise multiply adjustable clamp means, for example, securement to couch, vertical adjustment, angular adjustment, etc., in order to set a position and prevent rail movement relative. e.g., to the plane of the couch and thereby provide consistent and precise positioning of the rails. Therefore, indexing rails of the present disclosure provide repeatable device positioning, and in turn, support repeatable and accurate subject treatment.

An indexing rail of the present disclosure may be a single, unitary element, a multi-section (split) element corresponding, for example, to the upper and lower section of a treatment table, or be comprised of a plurality of sections, wherein each rail or section of rail may be independently secured to the couch, or alternatively, the sections may be engaged and secured (e.g., interlocked) and secured as a single unit to a couch.

Generally, rails of the present disclosure have at least top surface and bottom surface, and a plurality of indexed apertures therethrough, through which the at least one support arm component may be inserted from one side of an aperture through to the other, e.g., top to bottom of the aperture, and secured therein. Indexing rails of the present disclosure are, preferably, removably secured in a parallel plane and lengthwise to a treatment table or couch, though indexing rails may also be attached widthwise, or width- and lengthwise, as desired. A preferred attachment means comprises a clamping system for removably attaching at least one indexing rail, wherein the clamping system is configured to be adjustable vertically, horizontally, rotationally, or a combination thereof, and preferably finger activated.

Indexing rails may comprise multiple, e.g., one or more, indexed apertures for the insertion and securement of the at least one support arm. In some embodiments, the apertures may be smooth bores through which the first fixed end of the at least one support arm may be inserted therethrough and secured adjacent to the bottom surface of the rail via, e.g., a known pinning, capping, or other locking element. Alternative embodiments of the rail apertures may include splined or threaded apertures, or a combination thereof, useful for the indexed rotation or screwing in of the at least one support arm, respectively. In some embodiments, the indexed aperture may be adapted for rotational indexing, for example, a spline aperture configuration at predetermined angles of rotation, e.g., 1-90°, 2-50°, 3-30°, preferably 2-20°, and more preferably 5-10°, and corresponding to a first fixed end of a support arm configured with corresponding spline configuration angles and a shoulder stop. Such configuration enabling indexing rail engagement of the first fixed end of the arm via insertion into the spline aperture in a predetermined position and disengagement and rotation from that position to another predetermined and stable position. Thus, the first fixed end of the arm may be rotated and indexed to occupy an adjacent or other position to adjust and secure the arm position which translates to a change in bar component and breast stabilization position. In some embodiments, the one or more rail indexed apertures may be threaded bores into which a correspondingly threaded first fixed end of a support arm may be engaged and secured.

### Support Arm Components

Support arm components comprising one or more curvilinear arms, preferably non-articulating, adapted for coupling to a radiation couch are provided. The arm or arms are preferably a component having a first fixed end, i.e., removably fixed to a radiation couch, and an opposite free end, and may optionally be configured for rotational indexing of the arm. For ease of understanding, the supports arms of the device are illustrated in specific configurations, e.g., tubular and parallel rails. However, the configuration and shape of support arms of the present device is not necessarily limited to such.

The first end of a support arm may be fixed, e.g., directly to a radiation couch or to an indexed couch rail, at a desired location while the second and opposite free end of the support arm may be positioned to arch over the body of a subject, and thus, provide a fixed support arm with which a subject's body part, i.e., breast, may be engaged and immobilized as described herein. A preferred concentric radii of the support arm(s) of the present device is less than or equal to about 70 cm, a measurement currently corresponding to the bore opening of a CT scanner. However, the concentric radii may be increased or decreased without affecting the functionality of the present device. The support arm's first fixed end may be adapted for indexing, for example, by fixed degrees employing a spline configuration disposed on the fixed end corresponding to one or more couch rail spline apertures. The second and opposite free end may terminate with a functional or non-functional element disposed thereon. For ease of understanding, the support arm component of the present disclosure is shown without a functional element, e.g., claw, camera, hooks, etc., mounted thereon. In some embodiments, the support arm component may further comprise indexing indicia for positional tracking.

In some embodiments, one or more curvilinear arms may comprise a single arcuate element configured such that each of the two ends of the support arm are configured to be removably fixed to the treatment couch or indexed couch rail(s).

In some embodiments, the one or more curvilinear arms may comprise indexed apertures therethrough, with or without cleat functionality, configured for receiving, for example, one or more tension cords or one or more tensioner assembly elements, or one or more bars comprised of at least one tension cord and at least one tensioner assembly element.

In some embodiments, the one or more curvilinear arms may comprise substantially parallel elements, indexed arm rails (side-by-side surfaces having substantially the same distance continuously between them), defining an equidistant curvilinear space there between and configured for receiving one or more tensioner assembly elements, one or more bars comprised of at least one tension cord and at least one tensioner assembly element, or a combination thereof, wherein the one or more tensioner assembly elements or one or more bars may be removably and moveably secured in a first position on an indexed arm rail such that the tensioner assembly may be loosened and moved to a second or other position on an support arm rail.

Support arm components of the present disclosure may be a unitary element or may be comprised of multiple segments, and thus, constructed of a plurality of smaller elements adapted for interlocking engagement. Interlocking functionality of elements, the linking or joining of parts, is well known and may be achieved on the present device by any such functionality now known or yet to be developed.

Embodiments of the support arm or arms may carry various indicia useful for repeatable and accurate positioning of the device and its components in relation to a subject. The one or more support arms may be constructed of materials that are safe in the intended environment of use and that will not interfere with the intended procedures. The one or more support arm components of the disclosed device may be constructed from, for example, urethane, carbon fiber, plastic, foam, polyethane, surlin. Kevlar, fiberglass, thermaplasturethane, thermaplatlastormere, tempered glass, polycarbonate, rubber, nitrile, butyl, neoprene, wax, acrylic like material (e.g., fictive), resin, silicone, vinyl, urethane methacrylate (carbon), PU, PVC, liquid plastic urethane polymer, epoxy, leather, polyol, polymid, urethanealuminum mixture, stainless steel, titanium, tantalum, platinum, or palladium, and the like, or composites, alloys, or mixtures thereof, and may further comprise a coating, all of which may be sterilized. Each of the materials may be relatively malleable or intractable, and thus, may be formed or shaped without causing flaws and hold their shape once manufactured. Methods for producing the support component of the device, include, for example, stamping, pouring, compression molded, bladder molded, extrusion, casting, injection molding, casting, pultrusion, bladder molding, sand casting, hand layup method, and the like, and combinations thereof. Making some of the embodiments of the device without metal may be beneficial for combination use with magnetic resonance imaging (MRI) or other known or yet to be known medical devices, procedures, and/or processes. The support arm components of the present disclosure may be manufactured to be sterilized and reusable, or alternatively, manufactured as a single use and disposable component.

### Bar Component

Some embodiments of the present device may employ bar components (“bars”) comprised of at least one tension cord and at least one tensioner assembly element for, e.g., detachable engagement of a breast engagement plat as described herein. Some embodiments may employ one or more partial bars, i.e., tension cords, detachably engaged directly from a breast plat to a support arm component.

In their broadest terms, bars functionally couple a support arm component to a breast engagement plat component. Thus, in some embodiments, a bar may be comprised of a tension cord having a first hooked end (i.e., a first end of a tension cord having a hook element disposed thereon) and a second and opposite free end, wherein the free end may be engaged with a tensioner assembly element and the first hooked end is configured for detachable engagement with the at least one breast engagement plat. In some embodiments, a bar may be comprised of a tension cord having a first hooked end and a second and opposite free end, wherein the free end may be engaged with a support arm component. By way of example, one or more cleats disposed on the external surface of a tensioner assembly element mounted on a support arm component may be detachably engaged by the second and free end of a tension, thus, securing the placement of the tension cord while possessing the ability to re-position and re-cleat the tension cord any number of times. Tensioner assembly elements are well known and may be, for example, spring actuated, push button actuated, clam shell style, spooling, cleated, or other known or yet to be known configurations, provided the tensioner assembly element may be adapted for secure positioning on a support arm component of the present device. As used herein, the term “cleat” refers to a support or check for holding something in place, for example, a flange or wedge-shaped piece fastened to, projecting from, or incorporated into something, a configured aperture, or a configured fitting, each serving as a support, check, position hold, grip, securement, or attachment point.

In some embodiments, the at least one tensioner assembly element may comprise at least one cleat element into which the second free end of a tension cord may be secured. In some embodiments, the at least one tensioner assembly element may comprise a spool onto which the second free end of a tension cord may be threaded for winding adjustment. In some embodiments, the at least one tensioner assembly element may comprise at least one cleat element and at least one spool element. Cleat and spool tensioner assembly elements are well known and may be configured to receive, for example, a tension cord, secure the tension cord in place, and release the tension cord for repositioning and securement in one or more different positions. As used herein, the term “tensioner” as used with tensioner assembly elements, is intended to refer to an assembly useful, inter alia, to set and keep tension on an article, i.e., a cord, belt, wire, thread, or the like. Tensioner assemblies and their variables are well known in the art.

Some embodiments of bars may comprise a tension cord having a first hooked end for detachable engagement with at least one breast engagement plat and a second engagement end configured to directly engage a support arm aperture, for example, by insertion therethrough and securement, e.g., cleated. In some embodiments, the second engagement end may be threaded and correspond with one or more correspondingly threaded support arm apertures. In some embodiments, the tension cord second engagement end may be configured to be inserted through one or more support arm apertures and secured adjacent to the opposite surface. i.e., aperture exit, of a support arm aperture via, e.g., a known pinning, capping, or other locking element.

In some embodiments, the one or more support arms are individually configured for receiving one or more tensioner assemblies or one or more bars comprised of at least one tension cord and at least one tensioner assembly element, wherein the one or more tensioner assembly elements may be removably and moveably secured in a first position on an support arm such that the one or more tensioner assembly elements may be loosened and moved to a second or other position on the support arm.

Embodiments of the one or more tension cords may be constructed to be flexible or inflexible (rigid) from various materials. Thus, the various components of the bars of the present disclosure, cords and tensioner assemblies, may be constructed from urethane, carbon fiber, plastic, foam, polyethane, surlin, Kevlar, fiberglass, thermaplasturethane, thermaplatlastormere, tempered glass, polycarbonate, rubber, nitrile, butyl, neoprene, wax, acrylic like material (e.g., fictive), resin, silicone, vinyl, urethane methacrylate (carbon), PU, PVC, liquid plastic urethane polymer, epoxy, leather, polyol, polymid, urethanealuminum mixture, stainless steel, titanium, tantalum, platinum, or palladium, and the like, or composites, alloys, or mixtures thereof, and may further comprise a coating, all of which may be sterilized or configured in a strand, i.e., cord, or tensioner assembly element. Each of the materials may be relatively malleable or intractable, and thus, may be formed or shaped without causing flaws and hold its shape once manufactured. Methods for producing the support and/or one or more bars of the device, include, for example, stamping, pouring, compression molded, bladder molded, extrusion, casting, injection molding, casting, pultrusion, bladder molding, sand casting, hand layup method, and the like. Making some of the embodiments of the bars of the device without metal may be beneficial for combination use with magnetic resonance imaging (MRI) or other known or yet to be known medical devices, procedures, and/or processes.

### Breast Engagement Plat Component

The breast engagement plat component of the present disclosure may, for example, be configured as a sheet of material having a top surface and bottom surface, comprising a plurality of rigid or flexible bar engagement elements (“loops”) disposed on the top surface, indicia optionally disposed adjacent thereto (to facilitate, e.g., platting, mapping, tracking), and a bottom surface configured to couple to the breast of a radiation treatment subject, for example, with an adhesive element disposed on the bottom surface, the subject's breast, or a combination thereof. Manufacturing of breast engagement plats may include processes now known in the art and those yet to be developed, for example, laminated, heat treated, thermal formed, die cut, etc. In some embodiments, the breast engagement plat may be comprised of one or more layers of material, e.g., a bottom engagement layer, a center loop layer having top oriented loops, and a top stabilizing layer having a plurality of loop corresponding apertures through which the loops protrude (thereby stabilizing), and wherein the layers are bonded, e.g., heat-bonded, to create a single breast engagement plat component having the desired characteristics of stability, engaging loops, and the ability to couple to a subject breast.

In some embodiments, a single sheet of material having a top surface and bottom surface, configured with a plurality of paired parallel cuts, e.g., equivalently sized pairs, therethrough effectively creating bar engagement loops, rigid or flexible, either by manufacturing process or when functionally engaged by a bar tension cord first hooked end, and configured to, for example, have adhesive disposed on the bottom surface or engage adhesive for adhering to a subject's breast is provided. The plurality of paired parallel cuts may be the same size, various sizes, at various angles, or in various positions, provided the stability of the plat is not impaired by the size or position of the plurality of paired parallel cuts.

Embodiments of breast engagement plats may further comprise one or more types of indicia to facility, e.g., platting, mapping, tracking, identification, and other purposes. Breast engagement plats and bar tension cords of the present disclosure may be configured to interface with any now known or yet to be discovered stabilizing assembly, e.g., support arm assembly, braces, hoops, etc.

The instant device may be configured as a whole and complete singular device or system, or alternatively, may be configured as individual, replaceable, or substitutable components, any of which may be configured to engage with or removably attach to treatment tables, couches, or support boards. Radiation of breasts, in particular those categorized as large, is generally administered from the side(s) using specifically determined radiation beam angles. Because the heart and lungs lie directly beneath the breast, by immobilizing and lifting the individual breast off of and away from the thorax of a subject, the angle of the radiation beam during administration more effectively penetrates the breast tissue while concurrently avoiding irradiating organs, e.g., the heart and lungs. In its most basic form, the instant device immobilizes a breast and moves it away from the thorax, i.e., lifts the breast, of a subject being treated and stabilizes it in a specific and repeatable position. Such immobilization decreases radiation exposure of the organs of the subject, increases the accuracy of the radiation treatment, produces accurate and reproducible treatment(s), and reduces radiation treatment side effects, all while allowing the subject to remain in relative comfort during treatment.

This device allows large and/or pendulous breasts to be repeatably and consistently positioned for accurate treatment. For the reasons discussed herein, large breasted subjects in need of radiation treatment and other procedures may be denied the preferred one-week long partial breast irradiation (as opposed to 3-4 weeks of whole breast treatment). Thus, the present device may, via its accuracy and repeatability of treatment and reduced side effects, reduce or eliminate breast shrinkage resulting from whole breast treatment due to sensitivity of the fat cells in the breast, and in turn, reduce or eliminate related breast asymmetry in relation to the contralateral (untreated) breast following radiation. The disclosed device allows for the targeting of a small area, currently challenging in large breasts, decreasing and/or eliminating side effects of whole breast radiation treatment.

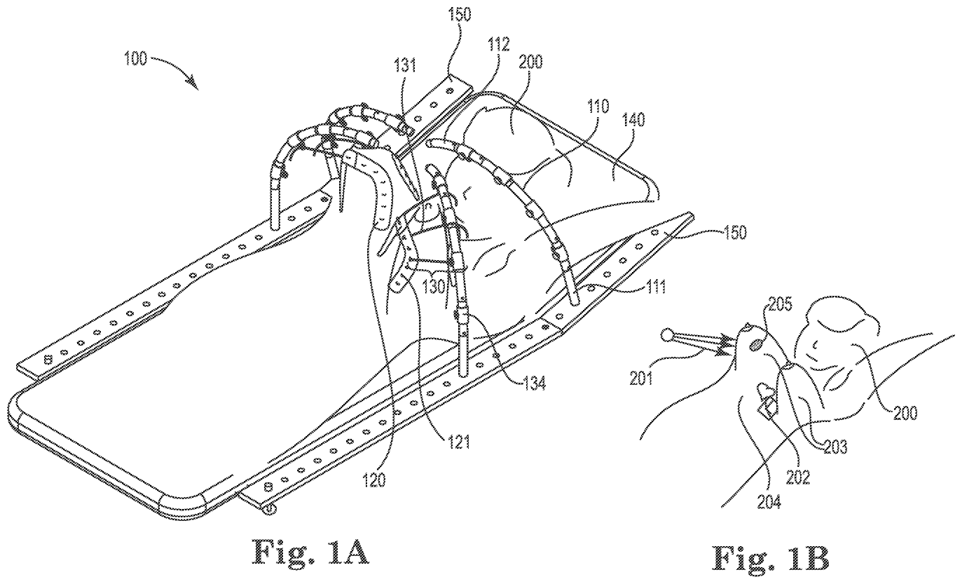

Referring now to FIG. 1A, a perspective view of an exemplary preferred embodiment of the breast immobilization device 100 comprising at least one indexing couch rail 150 mounted on opposite lengthwise sides of a radiation couch 140 to which at least one support arm component 110 comprising a generally curvilinear configuration having a first fixed end 111 and opposite second free end 112 is detachably fixed; at least one breast engagement plat 120 having at least one engagement loop 121 disposed thereon: and at least one bar component 130 comprising at least one tension cord 131 and at least one tensioner assembly element 134, wherein the bar component 130 functionally and removably couples the breast engagement plat 120 to the arm support 110, is shown. FIG. 1B shows a perspective view of a subject 200 receiving radiation therapy from multiple radiation beams 201 to a breast immobilized by the present device 100 without targeting the subject's 200 internal organs 202. For ease of understanding, the figures is an isolated view of the subject 200 without the components of the device 100 overlaid. Absent immobilization and lift of the breast or breasts 203 of a subject 200, the breast 203 may fall to the side of the subject and toward the axilla, making it difficult at best, and sometimes impossible, to provide radiation therapy to a subject without involving the heart and/or lungs 202 to some degree, even with DIBH (Deep Inspiration Breath Hold). The present device 100, as described and shown herein, lifts a subject's entire breast 203 away from the thorax 204 and organs 202 of the subject 200 and positions it for radiation, including radiation from the side. The device 100, when engaged, produces an accurate and reproducible breast position for treatment. Thus, as radiation is typically delivered while the patient is in the supine position on a radiation couch 140, the device disclosed herein prevents the subject's breast from falling laterally toward the axilla or down toward the abdomen. Further, the device 100 may be utilized to support one or both breasts 203, or alternatively, utilized to immobilize the contralateral breast—not the intended target of radiation therapy—thereby removing it from the field of radiation.

In some embodiments, alternative markers, indicia, and/or sensors, may be attached to or used in conjunction with the device 100, the individual device components, and/or the subject 200. By way of example, the breast 203 of a subject 200 may be positioned and semi-permanent or permanent indicia affixed to the epidermis of the subject 200, and light beams emitting from various positions in, e.g., the wall, may be indexed to the epidermis indicia Because large or pendulous breasts may alter position from day to day, the use of such indicia and indexing provides a more focal radiation to, for example, a partial breast or lumpectomy cavity 205.

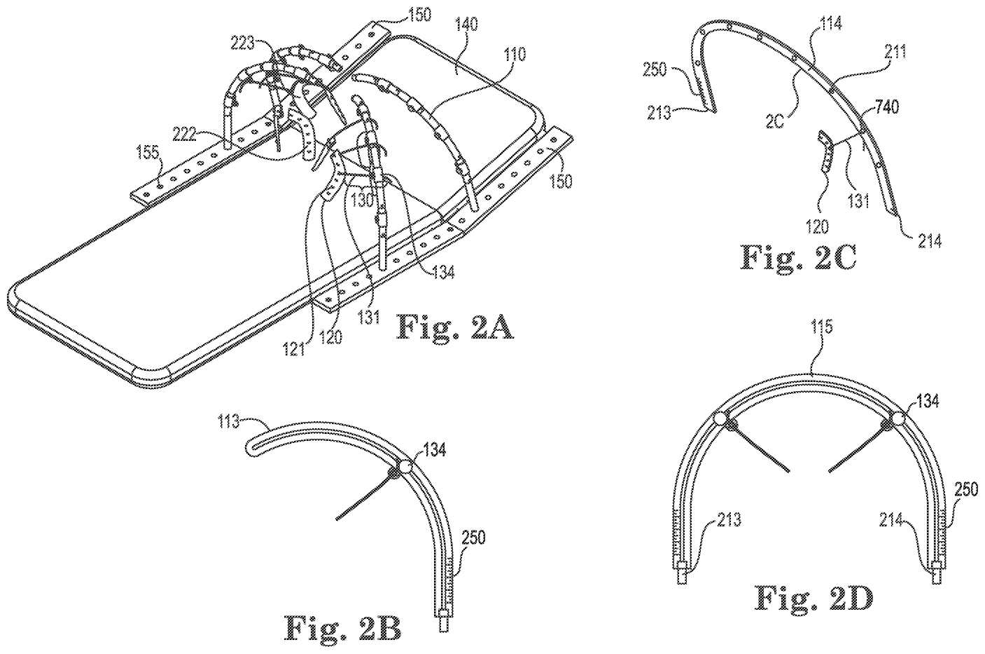

FIGS. 2A-D show exemplary embodiments of the present device. The support arm component 110 of the device may be attached to a couch, table, or board 140 using now known or yet to be discovered attachment means, methodology, and configurations. The support arm component 110 may, for example comprise a curvilinear arm, hoop, arch, semi-circle, C-shape, or the like, provided that it is of sufficient size, i.e., width and breath, to extend over a subject and attach to a table. In some embodiments, the support component 110 may be configured in a shape with no acute or obtuse angles, or alternatively, one or more angles, for example, one or more 90 degree angles. Myriad alternative configurations of a support arm component fulfilling the functionality disclosed herein are possible. While the description of the support component 110 of the present device set forth is exemplary and carries advantages, alternative configurations of support components may be provided or contemplated within the scope of this disclosure. FIG. 2A illustrates an embodiment of the device (see, FIG. 1A) without the inclusion of a subject for ease of understanding. The device 100 comprises one or more couch indexing rails 150 having a plurality of rail indexing apertures 155 therethrough, wherein the one or more indexing rails may be mounted on opposite sides of a couch 140 and to which at least one support arm component 110 comprising a generally curvilinear configuration having a first fixed end 111 and an opposite second free end 112 is detachably fixed; at least one breast engagement plat 120 having a top surface 222 with at least one engagement loop 121 disposed thereon and a bottom surface 223 configured to adhere to a subject's breast; and at least one bar component 130 comprising at least one tension cord 131 and at least one tensioner assembly element 134 movably mounted on the at least one support arm 110, wherein the bar component 130 functionally and detachably couples the at least one breast engagement plat 120 to the at least one support arm 110. In some embodiments, the at least one support arm and/or the at least one couch indexing rails may further comprise indexing indicia (not shown).

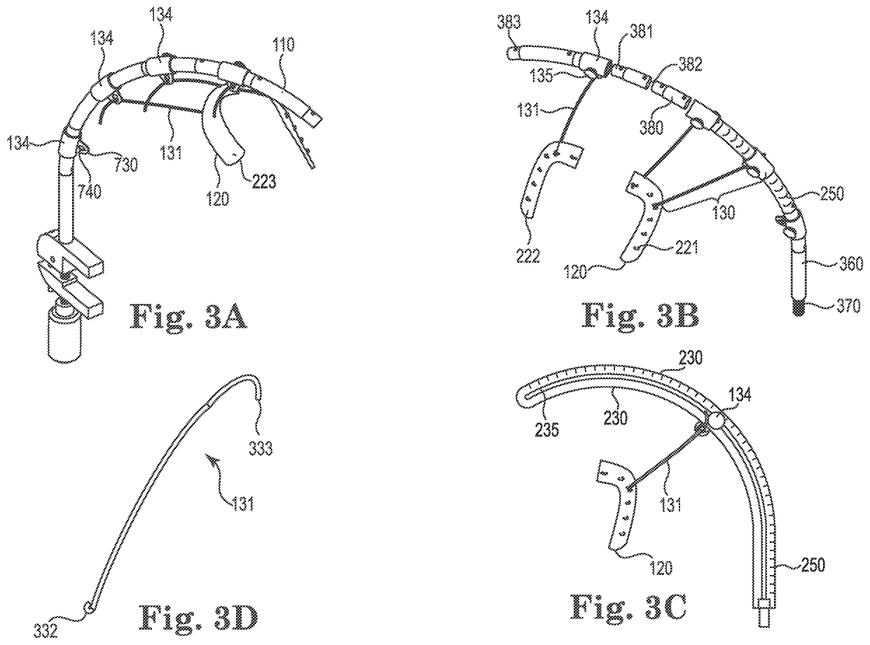

In another exemplary embodiment, FIG. 2B, a support arm of the device as described herein is illustrated, wherein the at least one support arm 113 having a first fixed end 111 and an opposite second free end 112 is comprised of at least two substantially parallel curvilinear arm rails 230 (FIG. 3C) defining an equidistant curvilinear space there between 235 (FIG. 3C), configured for receiving one or more tensioner assembly elements 134, e.g., through which a tensioner assembly element 134 may be inserted and secured, and optionally further comprising indexing indicia 250.

FIG. 2C provides a support arm exemplary embodiment of the device as described herein, wherein the at least one support arm 114 comprises a generally curvilinear configuration having two opposite fixed ends 213, 214, and further comprising one or more apertures 211 therethrough in the plane perpendicular, parallel, or a combination of perpendicular and parallel to the treatment table 140, the one or more apertures 211 optionally configured with a cleat 740 to secure, e.g., a tension cord 131 coupled to a breast engagement plat 120, and further optionally comprising indexing indicia 250 corresponding to the one or more support arm apertures 211 or discrete positions on the support arm 114.

FIG. 2D illustrates yet another exemplary embodiment wherein a support arm component 115 comprising a generally curvilinear configuration having two opposite fixed ends 213, 214, comprised of at least two substantially parallel curvilinear arm rails 230 (FIG. 3C) defining an equidistant curvilinear space there between 235 (FIG. 3C), configured for receiving one or more tensioner assembly elements 134, e.g., through which a tensioner assembly element 134 may be inserted and secured, and optionally further comprising indexing indicia 250.

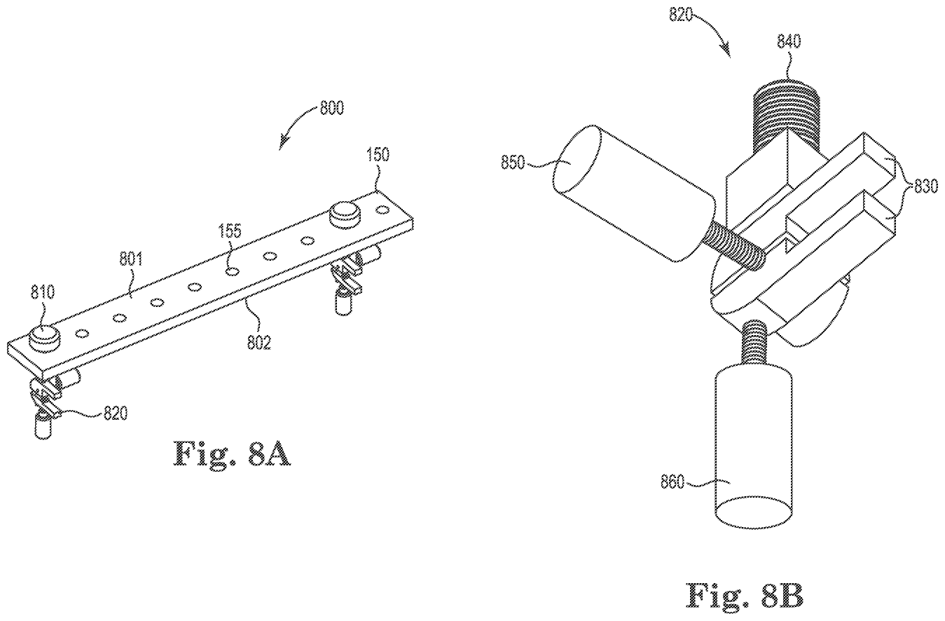

Moving now to FIGS. 3A-D, isolated views of a support arm 110, 113 of the device having at least one bar 130 (comprised of at least one tension cord 131 and at least one tensioner assembly element 134) mounted thereon and functionally engaged with at least one breast engagement plat 120 are shown. FIG. 3A shows a first side view of a support arm 110 having mounted thereon a plurality of bars 130 comprised of tubular tensioner assembly elements 134 having a wedge 730 defining a cleat 740 extending outwardly therefrom and a hooked tension cord 131, wherein the at least one breast engagement plat 120 is functionally engaged by the first hooked end 332 (not shown) of the at least one tension cord 131, and wherein the second free end 333 of the at least one tension cord 131 is engaged with a cleat 740 of a corresponding at least one tensioner assembly 134. As shown, the first fixed end 111 of the support arm 110 may optionally be configured, e.g., with a clamping means (see, FIG. 8B for detail), for detachably fixation directly with a treatment couch 140.

FIG. 3B shows a second and opposite side view of a support arm 110 having mounted thereon a plurality of bars 130 comprised of tubular tensioner assembly elements 134 as shown in FIG. 3A, comprising a spring actuator button 135 for allowing tensioner assembly element 134 release, movement, and securement along the path of the support arm 110 when actuated. The support arm may further and optionally comprise positional indicia 250. FIG. 3B further exemplifies a support arm component comprised of segments 380 having a male end 381 and female end 382 adapted for interlocking engagement via, for example, insertion of the male end 381 into the female end 382 of another segment 380, a locking means 383, and a generally straight segment 360 having a splined first fixed end 370.

FIG. 3C shows a side view of the support arm 113 (shown in FIG. 2B) comprised of at least two substantially parallel curvilinear arm rails 230 defining an equidistant curvilinear space there between 235, and configured for receiving one or more tensioner assembly elements 134, e.g., through which a tensioner assembly element 134 may be inserted and secured, and having mounted thereon at least one bar 130 comprised of at least one spooling tensioner assembly element 134 and at least one hooked tension cord 131 having a first hooked end 332 and a second free end 333, wherein the second free end 333 is functionally engaged with the tensioner assembly element 134, and at least one breast engagement plat 120 functionally engaged by the first hooked end 332 of at least one tension cord 131. A preferred embodiment of a tension cord of the present device are shown in FIG. 3D, wherein the tension cord 131 is comprised of a first end having a hooked disposed thereon 332 and a second free end 333 which may be, for example, cleated into a tensioner assembly, threaded into a tensioner assembly, spooled onto a tensioner assembly, spooled onto and cleated into a tensioner assembly, or combinations thereof. Each of the support arm components of the present device 100 may comprise the same or different diameter(s), length(s), a combination of multiple diameter(s) and/or length(s), or may be adjustable with respect thereto.

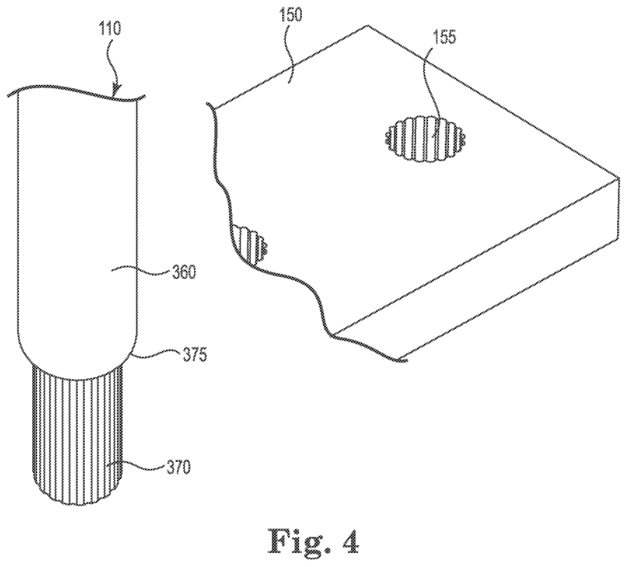

As shown isolated in FIG. 4, in some embodiments, a generally straight segment first fixed end 360 of a support arm 110, 113 may comprise a splined or indexed spline insertion element 370 on the terminating end having the same (not shown) or smaller diameter as the body of the support arm 110, 113 and a shoulder 375 for indexable engagement with a spline aperture 155 of a couch rail 150. Upon insertion of the straight segment 360 spline insertion element 370 into the spline aperture 155, the shoulder 375 acts as a stop for the support arm 110, 113 thereby positionally setting the support arm 110, 113 and allowing for support arm removal, rotation, and reinsertion into the spline aperture 155 for rotational indexing without, for example, sacrificing vertical positioning of the support arm 110, 113.

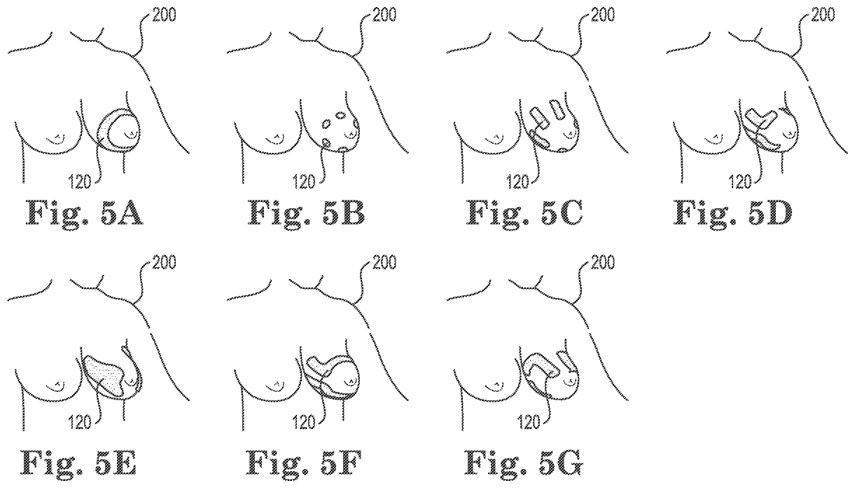

Moving to FIGS. 5A-G, multiple, non-limiting, exemplary configurations of breast engagement plat components 120 of the disclosed device are shown. Based on a subject's 200 build, chest circumference, breast size, etc., one or more breast engagement plats 120 of the disclosed device 100 may be semi-custom fit to each subject 200 for optimal performance: by adhering one or more configurations of breast engagement plats 120 to various positions on a subject's breast 203, myriad immobilization positioning and customization based on the physique or physical attributes of a subject 200 is possible with the present device. Independent of the shape or size of the at least one breast engagement plat 120, the plats 120 carry essentially the same attributes, including, for example, one or more bar engagement elements or loops 121 disposed on a top surface 222 of the breast engagement plat 120 configured for tension cord 131 engagement and a bottom surface 223 configured for coupling with, e.g., adhering to, the breast of a subject 200.

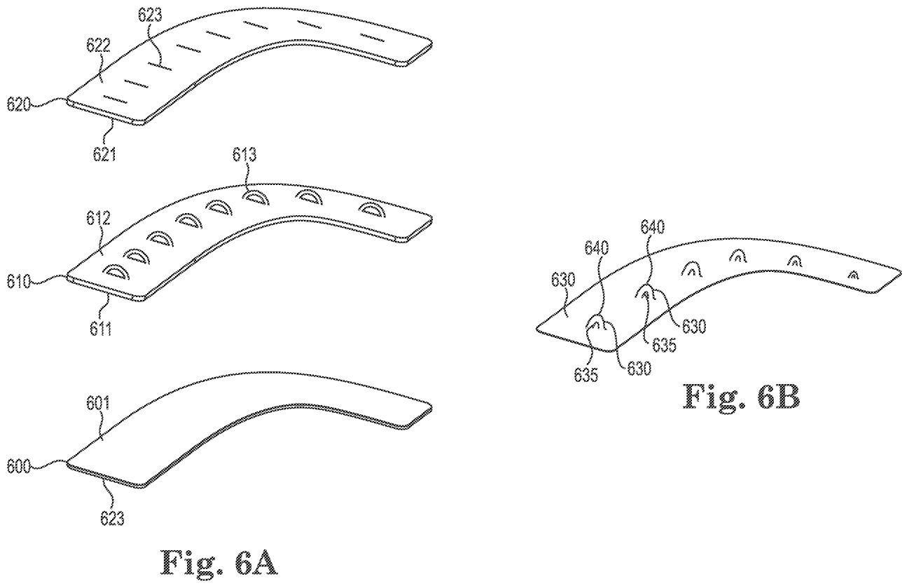

FIGS. 6A-B show isolated and exploded views of preferred exemplary construction of breast engagement plats 120 of the present device. Broadly, as shown in FIG. 2A, breast engagement plats 120 comprise at least a top surface 222 having at least one and preferably a plurality of bar engagement elements 121, e.g., clasps, hooks, catches, holders, links, locks fasteners, grips, loops, or the like, collectively referred to as “loops” disposed on or protruding therefrom for detachable engagement with a hooked tension cord 131, and bottom surface 223 configured for coupling with the breast of a subject, e.g., having disposed thereon an adhesive for receiving and removably attaching to a subject's breast 203 or comprising a smooth, flat surface for removably engaging with adhesive disposed on a subject's breast 203.

FIG. 6A provides an exemplary multiple layer breast engagement plat 120 comprising, for example, three layers of construction material, wherein a first or bottom layer 600 comprises a bottom surface 623 configured for receiving adhesive in any manner now know or known in the future or for adhering to a breast with adhesive disposed thereon, and a top surface 601 for engagement with a second or middle layer 610. The second or middle layer 610 comprises a bottom surface 611 configured for engagement with the top surface 601 of the bottom layer 600 and a top surface 612 having at least one loop 613 disposed thereon, and a third or top layer 620 comprising a bottom surface 621 configured for engagement with the top surface 612 of the second layer 610 and top surface 622, the top layer 620 having at least one elongated loop corresponding aperture 623 therethrough for receiving the one or more loops 613 during assembly (when the at least one loop 613 is inserted therethrough). Each of the layers. 600, 610, 620 of a multi-layered breast engagement plat 120 of the present device may each be comprised of the same or different construction material. Bonding of layered material is known in the art, and any of the now known or yet to be known methodologies may be employed to form a single plat from multiple layers.

FIG. 6B is provides an exemplary breast engagement plat 120 comprised of single layer of construction material 630 into which parallel or paired slits 634, 635, are, for example, die cut and thermally formed, effectively giving rise to at least one loop 640, preferably a plurality thereof, for functional engagement by a hooked end 332 of tension cord 131. Depending upon the construction material, the at least one loop 640 may receive further treatment, e.g., chemical, heat, etc., to, for example, harden, strengthen, shape, etc., the at least one loop 640.

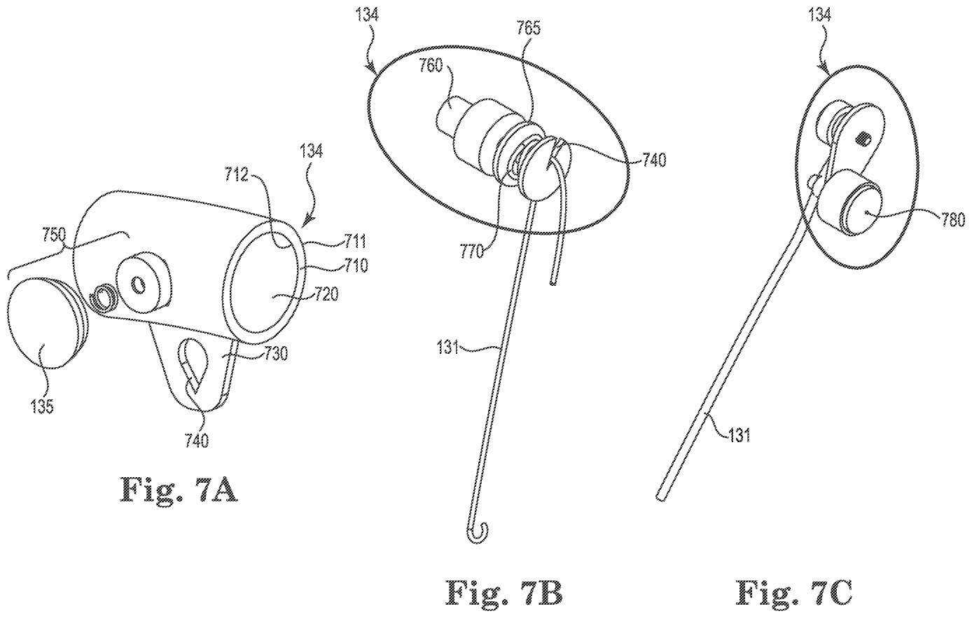

Turning to FIGS. 7A-C, exemplary embodiments of tensioner assembly elements are shown, however, it is noted that any known tensioner assembly now known or known in the future may be adapted for use with the present device. A first and preferred embodiment of a tensioner assembly 134 is shown FIG. 7A comprising a tube 710 having an outside tube wall 711 and inside tube wall 712, wherein the inside wall defines a tubular aperture 720 configured to have a diameter corresponding to functionally engage with and receive a support arm 110, 114 of the present device therethrough, and further comprising a wedge 730 extending in a generally perpendicular position to and from the outside tube wall, wherein the wedge defines a cleat 740 for receiving and securing, e.g., a tension cord 131. A tension cord 131 may be lengthened or shortened to adjust breast position, by uncleating a secured tension cord 131 from a cleat 740, adjusting its length as desired, and re-cleating. The tensioner assembly 134 may further comprise a tensioner assembly release button 135 and spring assembly 750 for enabling positioning, movement, and repositioning of the tensioner assembly 134 longitudinally along a support arm 110, 114. By engaging the release button 135, moving the tensioner assembly 134 to a second or other position on the support arm 110, 114 and disengaging the release button 135, the tensioner assembly 134 is secured in the second or other position.

FIG. 7B shows an exemplary embodiment of a spooling and cleated tensioner assembly element wherein a tensioner assembly back 760 may be inserted through an arm rail aperture 235 of a support arm 113, 115 and functionally coupled with, e.g., interlocking engagement, a tensioner assembly front 765 comprised of a spool 770 and cleat 740 assembly for manually engaging (spooling the tension cord 131 onto the spool 770 of the tensioner assembly 134 to lengthen or shorten the tension cord), securing (inserting the free end of the tension cord 131 into the cleat 740), and adjusting the length a tension cord 131 (engaging the rotational functionality of the spool). By disengaging, either partially or fully, the tensioner assembly front 765 from the tensioner assembly back 760, the tensioner assembly element 134 may be positionally adjusted on the support arm 113, 115 by longitudinal movement along the parallel rails 230 of the support arm 110.

FIG. 7C is another exemplary embodiment of a spooling tensioner assembly element 134 further comprising a button spring actuated spooling element 780 to release and adjust a tension cord 131. A multitude of tensioner assembly elements are known and may be adapted for use in conjunction with the present device.

As described, the one or more support arms 110, 113, 114, 115, of the present invention may be detachably engaged directly with a radiation couch 140, for example, via configuring the first fixed end 111 or two fixed ends 213, 214 with a clamping means disposed thereon (see, FIG. 3A). Alternatively, in preferred embodiments, the one or more support arms 110, 113, 114, 115 of the present invention may be detachably engaged with the one or more radiation couch rails 150 detachably fixed, mounted, to a couch 140. An attachment means for mounting one or more couch rails 150 to a couch 140 may include, for example, clamps, screw clamps, cams, plackets, combinations thereof, or the like. Moving to FIGS. 8A-B, a rail engagement system 800 for stable and detachable engagement of an indexed couch rail 150 with a radiation couch 140 (not shown) is provided. The rail engagement system 800 comprised at least one couch rail 150 having at least a top surface 801 and a bottom surface 802, a plurality of rail apertures 155 therethrough, e.g. substantially vertically bored therethrough, at least one clamping mechanism 820 (see, FIG. 8B) comprising a threaded top member 840 disposed thereon, and a correspondingly threaded clamp cap 810. The at least one couch engagement clamp 820 may be secured to the couch rail 150 by inserting the threaded top member 840 through a couch rail aperture 155 and threading and tightening the corresponding threaded clamp cap 810 thereon. The rail 150, clamp 820, and clamp cap 810 may be engaged and secured in various configurations. For ease of understanding, a couch engagement clamp 820 is shown in and described a “bottom up” configuration (clamp 820 inserted upwardly through a rail aperture 155 from the bottom surface 802 of the couch rail 150 and secured with a clamp cap 810 adjacent to the top surface 801 of the couch rail 150); a top down or opposite configuration wherein a clamp 820 may be inserted downwardly through rail aperture 155 from the top surface 801 of the couch rail 150 and secured with a clamp cap 810 adjacent to the bottom surface 802 of the couch rail 150 may be employed as well.

As shown in FIG. 8B, the at least one couch engagement clamp 820 may be coupled to a couch 140 for example by loosening (unscrewing) a clamp vice adjustment knob 860 to open the clamp vice arms 830, engaging the clamp vice arms 830 with the couch 140, and tightening the adjustment knob 86i0 to secure the rail 150 to the couch 140. The couch engagement clamp 820 may further an optionally comprise additional adjustments, e.g., a clamp angle adjustment knob 850 for adjusting the angle of the couch rail 150 relative to the plane of a couch 140.

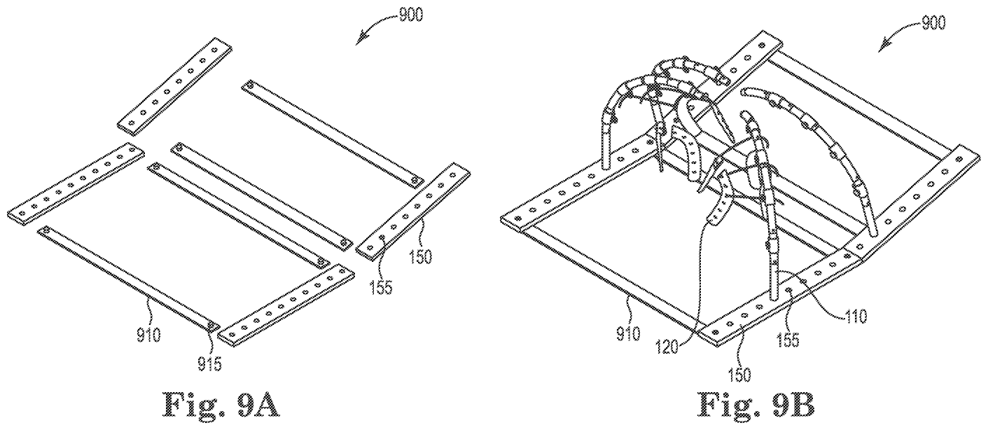

Turning finally to FIGS. 9A-B, an exemplary modular couch rail component embodiment 900 is provided. As shown in FIG. 9A, the couch rail module 900 comprises at least one indexing rail 150 mounted on a frame comprised of a plurality of crossbars 910 having at least one rail engagement element 915, e.g., a peg, disposed thereon for detachable, semi-permanent, permanent, or a combination thereof, engagement with a rail aperture 155. When assembled, FIG. 9B, the rail module 900 may be positioned atop a couch 140, overlaid, without being detachably mounted to the couch, and positioned to engage with the remaining components of the device as disclosed herein.

### In Use

As shown in the attached figures, the presently disclosed device positions a breast for radiation beam application from multiple angles without direct internal organ radiation exposure. With a subject 200 in the supine position on a table or couch 140 with their arms, e.g., raised above their head, and wearing a breast engagement plat component 120, a support component 110 is detachably secured to at least one couch rail 150 and positioned arching over the subject 200. Next, at least one bar component 130 functionally coupled to the plat component 120 is engaged with the support component 110, and optionally adjusted to optimize breast position for radiation treatment. Without the use of a breast immobilization device 100, radiation treatment, i.e., radiation beams 201, is delivered, even incidentally, to both the breast 203 and the internal organs 202 of a subject. In a secure and immobilized breast position, a subject 200 may receive radiation from the side of the breast or any chosen radiation beam angle 201, e.g., an optimized angle, without involvement of the internal organs 202.

The specific methodology of use of the presently disclosed device may vary, for example, the order of the steps, but a preferred method consists of the steps of: securing at least one indexing rail 150 to a radiation couch 140; adhering at least one breast engagement plat 120 having at least one loop 121 disposed thereon to a subject 200; arranging the subject 200 in a supine position on the radiation couch 140: securing at least one support arm 110, 113, 114, 115 to the at least one indexing rail 150; mounting at least one tensioner assembly element 134 on the at least one support arm 110, 113, 114, 115; detachably coupling at least one first hooked end 332 of a tension cord 131 to the at least one loop 121 disposed on the breast engagement plat 120; and adjustably coupling the opposite and free end 333 of the tension cord 131 to the at least one mounted tensioner assembly element 134.

Another method consists of the steps of: adhering at least one breast engagement plat 120 having at least one loop 121 disposed thereon to a subject 200; arranging the subject 200 in a supine position on a radiation couch 140; securing at least one support arm 110, 113, 114, 115 to the couch; mounting at least one tensioner assembly element 134 on the at least one support arm 110, 113, 114, 115: detachably coupling at least one first hooked end 332 of a tension cord 131 to the at least one loop 121 disposed on the breast engagement plat 120; and adjustably coupling the opposite and free end 333 of the tension cord 131 to the at least one mounted tensioner assembly element 134.

For embodiments employing one or more couch rails 150, mount a first clamp 820 onto a couch rail 150 by inserting the top threaded member 840 through a couch rail aperture 155 and secure therethrough by tightening the threaded clamp cap 810 thereon. Repeat the clamp mounting process with a second or more clamp 820. To fix the couch rail 150 to the couch 140, open the vice arms 830 of each mounted clamp 820, engage each clamp 820 with the couch 140 by fitting the couch side in between the vice arms 830, manually adjusting the rail 150 to the desired position and closing (tightening) the vice arms 830 of the clamp 820. Optionally, the angle of the rail 150 with relation to the plane of the couch 140 may be adjusted with angular adjustment knob 850. Next, adhere at least one breast engagement plat 120 directly in contact with the breast 203 of a subject 200. Some embodiments of the breast engagement plats 120 are envisioned to have adhesive disposed on the bottom surface 223 for engagement with a subject 200; in other embodiments it is envisioned that adhesive may be dispersed on the breast of a subject 200 onto which a breast engagement plat 120 may be placed engaging the bottom surface 223 therewith. Position the subject 200 in a supine position on the couch 140 and insert and secure the first fixed end 111 of the at least one support arm 110, 113 into a couch rail aperture 155 and orient the support arm 110, 113 in an arcuate position over the subject 2W, or insert the two opposite fixed ends 213, 214 of support arm 114, 115 into couch rail apertures 155, one on each side of couch 140.

Embodiments in which a clamp means 820 is disposed directly on the fixed end 111, 213, 214 of a support arm 110, 113, 114, 115 mount the clamp 820 onto a couch 140 by opening the vice arms 830 of the clamp 820, engage each clamp 820 with the couch 140 by fitting the couch side in between the vice arms 830, manually adjusting the support arm 110, 113, 114, 115 to the desired position and closing (tightening) the vice arms 830 of the clamp 820. Optionally, the angle of the arm 110, 113, 114, 115 with relation to the plane of the couch 140 or in relation to the subject 200 may be adjusted with angular adjustment knob 850.

In embodiments employing a spline indexing means, the at least one support arm 110, 113 may be rotationally adjusted to a desired position by backing it out of the rail aperture 155, rotating and reinserting it back into the aperture 155. Next, position and secure at least one tension assembly element 134 on the at least one support arm 110, 113, 114, 115. Catch at least one loop 121 with the first hooked end 332 of a tension cord 131 and secure the second opposite free end 333 to a cleat of a tubular tensioner assembly element 134, or alternatively, cleat 740 the second opposite free end 333 directly to the arm support 114. For tension assembly elements employing spooling functionality, spool and secure the second free end 333 of a tension cord 131 to the tensioner assembly 134 before catching a loop disposed on a breast engagement plat 120. Once one or more bars 130 (including at least a tension cord and a tension assembly 134) is engaged and coupling the breast engagement plat(s) 120 to the support arm(s) 110, 113, 114, 115, immobilization and position adjustments may be made by disengaging the tension cord 131, lengthening or shorting, and re-engaging the tension cord 131. Alternatively, adjustment may be made by disengaging the tension assembly element 134, moving it along the path of the support arm 110, and re-engaging it at a different position.

Additionally and optionally, the step of positional adjustment of a subject's breast may comprise any number of adjustments of the device, including but not limited to: rotational indexing of at least one support arm 110, 113 of the device, tensioner assembly element 134 placement along the path of the at least one support arm, and, shortening or lengthening the at least one engaged tension cord 113.

A method of medical, e.g., radiation, treatment comprising the steps of immobilizing and supporting a subject's breast using any embodiment of the device described herein, and delivery of accurate and repeatable radiation to the subject is provided.

The disclosure provides an improved breast immobilization device useful for radiation therapy and/or other procedures. The device disclosed herein may be used, for example, to support and lift pendulous breasts for improved radiation therapy accuracy combined with reduced organ radiation exposure. That is, the present device may provide improved breast position, and in turn, reduce the irradiate volume of heart, lung, and other organs, and reduce the side effect of radiation treatment. Moreover, the device may be used in combination with a wide range of radiation delivery systems making it broadly applicable.

What is claimed is:

1. A device for immobilizing at least one breast of a radiation therapy subject, the device comprising:
at least one support arm component;
at least one breast engagement plat; and
at least one bar component detachably engaging the at least one support arm component to the at least one breast engagement plat.

2. The device according to claim 1, further comprising at least one radiation couch indexing rail.

3. The device according to claim 2, wherein
the at least one indexing couch rail comprising at least a top surface, a bottom surface, and a plurality of apertures therethrough, is secured lengthwise and in parallel plane to the radiation couch;
the at least one support arm component comprises a curvilinear arm having a first fixed end and a second free end, wherein the first fixed end is detachably fixed to the at least one indexing couch rail;
the at least one bar component comprises at least one tension cord and at least one tensioner assembly element, wherein the at least one tension cord has a first end having a hook element disposed thereon and a second and opposite free end, and the at least one tensioner assembly element is movably mounted on the at least one support arm;
the at least one breast engagement plat comprises a top surface and bottom surface, wherein at least one bar engagement element is disposed on the top surface; and
the first end of the at least one bar tension cord detachably couples to the at least one bar engagement element of the breast engagement plat and the second free end of the tension cord detachably couples to the at least one tensioner assembly element.

4. The device according to claim 3, wherein the plurality of indexing couch rail apertures comprise smooth bores, splined bores, threaded bores, or a combination thereof.

5. The device according to claim 3, wherein the support arm component comprises a non-articulating support arm.

6. The device according to claim 5, wherein the support arm component comprises a plurality of substantially parallel arm support rails defining a space therebetween adapted for receiving at least one tensioner assembly element.

7. The device according to claim 1, wherein the support arm component comprises two fixed ends.

8. The support arm component according to claim 7, further comprising a plurality of apertures therethrough.

9. The device according to claim 5 or 7, wherein the support arm component comprises a plurality of segments adapted for interlocking engagement.

10. The device according to any one of claims 3-9, further comprising indexing indicia.

11. The device according to claim 3, wherein the at least one breast engagement plat is comprised of a single layer or a plurality of layers of material.

12. The device according to according to claim 11, wherein the top surface comprises at least one bar engagement element disposed thereon and the bottom surface is configured to couple to the breast of a subject.

13. The device according to claim 3, wherein the at least one tension cord is adapted for direct engagement with the at least one support arm.

14. The device according to claim 3, wherein the tension assembly element is selected from spring activated, spooling, cleated, and spooling and cleated functionality.

15. The device according to claim 1, wherein
the at least one support arm component comprises a curvilinear arm having a first fixed end and a second free end, wherein the first fixed end is detachably fixed to a treatment couch;
the at least one bar component comprises at least one tension cord and at least one tensioner assembly element, wherein the at least one tension cord has a first end having a hook element disposed thereon and a second and opposite free end, and the at least one tensioner assembly element is movably mounted on the at least one support arm;
the at least one breast engagement plat comprises a top surface and bottom surface, wherein at least one bar engagement element is disposed on the top surface; and
the first end of the at least one bar tension cord detachably couples to the at least one bar engagement element of the breast engagement plat and the second free end of the tension cord detachably couples to the at least one tensioner assembly element.

16. A method of using a device for immobilization of a breast, the method comprising the steps of:
securing at least one indexing rail to a radiation couch;
adhering a breast engagement plat having at least one loop disposed thereon to a subject;
arranging a subject in a supine position on the radiation couch;
securing at least one support arm to the at least one indexing rail or to the radiation couch;
mounting at least one tensioner assembly element on the at least one support arm;
functionally engaging a tension cord having a first hooked end and second free end, wherein the first hooked end engages the at least one loop disposed on the breast engagement plat and the second free end is adjustably coupled to the at least one mounted tensioner assembly element.

17. The method according to claim 16, further comprising the step of adjusting the components of the device in relation to the subject.

18. A method of using a device for immobilization of a breast, the method comprising the steps of:
adhering at least one breast engagement plat having at least one loop disposed thereon to a subject;
arranging the subject in a supine position on a radiation couch;
securing at least one support arm to the radiation couch;
mounting at least one tensioner assembly element on the at least one support arm;
functionally engaging a tension cord having a first hooked end and second free end, wherein the first hooked end engages the at least one loop disposed on the breast engagement plat and the second free end is adjustably coupled to the at least one mounted tensioner assembly element.

19. The method according to claim 18, further comprising the step of adjusting the components of the device in relation to the subject.

20. A method of medical treatment comprising the steps of: immobilizing and supporting a subject's breast; and delivery of accurate and repeatable radiation to the subject's breast, wherein the exposure of the subject's organs to radiation is minimized.

21. A couch rail module comprising
at least one indexing rail having a plurality of rail apertures mounted on a frame comprised of a plurality of crossbars and having at least one rail engagement element disposed thereon.
